(19) United States
(12) Patent Application Publication
(10) Pub. No.: US 2003/0129744 A1
(43) Pub. Date: Jul. 10, 2003

(54) Bvdv virus-like particles

(76) Inventors: Tobias Schlapp, Koln (DE); Franciscus Antonius Maria Rijsewijk, Lelystad (NL)

Correspondence Address: JEFFREY M. GREENMAN, VICE PRESIDENT, PATENTS AND LICENSING, BAYER CORPORATION, 400 MORGAN LANE, WEST HAVEN, CT, 06516, US

(21) Appl. No.: 10/312,062

(22) PCT Filed: Jun. 15, 2001
(86) PCT No.: PCT/EP01/06738

(30) Foreign Application Priority Data
Jun. 27, 2000 (EP) 00113088.9

(51) Int. Cl. C12Q 1/70; A61K 39/12; C12N 7/00; C12N 5/06; C12N 7/01; C12N 15/00; C12N 15/09; C12N 15/63; C12N 15/70; C12N 15/74; C12N 5/00; C12N 5/02

(52) U.S. Cl. 435/320.1; 435/5; 435/235.1; 424/204.1; 435/325

(57) ABSTRACT

The present invention relates to bovine viral diarrhea virus (BVDV) virus-like particles, a polycistronic RNA and DNA corresponding thereto encoding a polyprotein of BVDV structural proteins that are sufficient to form BVDV virus-like particles, a viral vector encoding factors and structural proteins for the assembly of BVDV virus-like particles, a vaccine comprising BVDV virus-like particles, a diagnostic kit and methods for preparing BVDV virus-like particles.

The present invention relates to bovine viral diarrhea virus (BVDV) virus-like particles, a polycistronic RNA and DNA corresponding thereto encoding a polyprotein of BVDV structural proteins that are sufficient to form BVDV virus-like particles, a viral vector encoding factors and structural proteins for the assembly of BVDV virus-like particles, a vaccine comprising BVDV virus-like particles, a diagnostic kit and methods for preparing BVDV virus-like particles.

Bovine viral diarrhea virus (BVDV) is the etiological agent of bovine viral diarrhea in cattle and has a world wide distribution and a prevalence that can be as high as 90%.

BVDV is a member of the genus pestivirus of the Flaviviridae family [Horzinek (1991), Pestiviruses—taxonomic perspectives, Arch. Virology Suppl. 3, 55-65]. BVDV has a positive-stranded RNA genome of approximately 12.5 kilo bases kb), coding for one open reading frame which can be translated into one large polyprotein [Collet et al. (1988), Proteins encoded by bovine viral diarrhea virus: the genomic organisation of a pestivirus, Virology 165, 200-208]. A BVDV virion consists of a genomic RNA fitted into a nucleocapsid which is surrounded by an envelope containing glycoproteins.

The structural proteins, nucleocapsid protein (N) and three envelope glycoproteins (E^{ms} or gp48, E1 or gp25, and E2 or gp53) are found in this order close to the N terminal end of the polyprotein. All three glycoproteins are preceded by a signal peptide sequence and are liberated from the polyprotein in the lumen of the endoplasmatic reticulum (ER) by host signal peptidases.

For a related Flavivirus, namely hepatitis C virus, expression of a genome segment which only encodes the structural proteins is sufficient to form virus-like particles [Baumert et al. (1998), Hepatitis C virus structural proteins assemble into virus-like particles in insect cells, J. Virol. 72,3827-3836].

Bovine herpesvirus 1 (BHV-1) is the etiological agent of infectious bovine rhino-tracheitis (IBR), of infectious pustular vulvovaginitis (IPV) and infectious balanopostitis (IBP). BHV-1 is found in cattle all over the world with high prevalences.

BHV-1 is a member of the alphaherpesviruses. It has a double stranded DNA genome of about 136 kilo base pairs and codes for about 70. genes of which about 30 genes are non essential for the replication of the virus. A spontaneous mutant with a deletion of the non essential glycoprotein E (gE) gene is used in safe and efficacious marker vaccines [Kaashoek et al. (1994), A conventionally attenuated glycoprotein E-negative strain of bovine herpesvirus type 1 is an efficacious and safe vaccine, Vaccine 12,439-444].

Vaccines to protect against BVDV induced diseases are based on either inactivated BVDV strains, on subunit vaccines based on isolated BVDV glycoproteins, on attenuated BVDV strains [reviewed by Van Oirschot et al. (1999), Vaccination of cattle against bovine viral diarrhea, Vet. Microbiol. 64, 169-183], on DNA vaccines [Reddy et al. (1999), Comp. Immunol. Microbiol. Infect. Dis. 22, 231-246; Harpin et al. (1999), Vaccination of cattle with a DNA plasmid encoding the bovine viral diarrhea virus major glycoprotein E2, J. Gen. Virol. 80, 3137-3144], or on vector vaccines. Vaccines based on attenuated BVDV strains may cause immuno-suppression and in utero infections Roth & Kaeberle (1983), Suppression of neutrophil and lymphocyte function induced by a vaccinal strain of bovine viral diarrhea virus with or without administration of ACTH, Am. J. Vet. Res. 44, 2366-2372; Liess et al. (1984), Studies on transplacental transmissibility of a bovine virus diarrhea (BVD) vaccine virus in cattle, Zentrbl. Veterinärmed. Reihe B 31, 669-681] and may be less efficacious in the presence of maternal antibodies.

Vaccines based on inactivated BVDV or subunit vaccines are less efficacious because the antigens are not produced intracellularly and therefore less efficiently presented to the T cell compartment of the immune system. E.g. two out of three baculovirus BVDV E2 subunit vaccines did not protect against a foetal BVDV infection in a sheep model [Bruschke et al. (1997), A subunit vaccine based on glycoprotein E2 of bovine virus diarrhea virus induces fetal protection in sheep against homologous challenge, Vaccine 15, 1940-1945].

DNA vaccines and vector vaccines do not have this disadvantage but the presently used DNA and vector vaccines are only expressing part of the structural proteins and fail to form the more immunogenic virus-like particles. E.g. WO 95/12682 describes the expression of only BVDV E2 in the TK locus of a BHV-1 strain [cf. also Kweon et al. (1999), Bovine herpesvirus expressing envelope protein (E2) of bovine viral diarrhea virus as a vaccine candidate, J. Vet. Med. Sci. 61, 395-401].

BVDV shows antigenic variation and can be divided into several antigenic groups (BVDV IA and EB, and BVDV II). This antigenic variation is mainly based on sequence variations in E2 [van Rijn et al. (1997), Subdivision of the pestivirus genus based on envelope glycoprotein E2, Virology 237, 337-348].

Vaccines based on E2 of only one BVDV type are more restricted in their cross protection [Bolin and Ridpath (1996), Glycoprotein E2 of bovine viral diarrhea virus expressed in insect cells provides calves limited protection from systemic infection and disease, Arch. Virol. 141, 1463-1477] than vaccines that also express the less variable N, E^{ms} and E1 proteins [Elahi et al. (1999), Induction of humoral and cellular immune responses against the nucleocapsid of bovine viral diarrhea virus by an adenovirus vector with an inducible promoter, Virology 261, 1-7].

The present invention provides BVDV virus-like particles.

The term “virus-like particles” as used herein refers to particles composed of most or all structural proteins of a virus that have a large part of the structural characteristics in common with their infectious wild type counterparts. However, upon interaction with the host cell virus-like particles do not produce progeny viruses because the proper nucleic acid sequences are not present in these particles. In case of BVDV virus-like particles, the nucleocapsids may be empty or may contain irrelevant RNA, but their overall structure is like wild type nucleocapsids and the surrounding envelope contains the same transmembrane glycoproteins E1 and E2 and the same membrane associated protein E^{ms}, as are found in wild type BVDV.

In particular, the virus-like particles according to the present invention comprise the BVDV structural proteins N, E^{ms}, E1 and E2. The invention contemplates BVDV virus-like particles which can be derived from various naturally occurring BVDV strains such as BVDV type I A strains (represented by the NADL strain), BVDV type I B strains (represented by the Osloss strain), and BVDV type II strains (represented by the 890 strain) including the cytopathic strain 87-2552 [Reddy et al. (1995), Anti-genic differences between a field isolate and vaccine strains of bovine viral diarrhea virus, J. Clin. Microbiol. 33, 2159-2161] and BVDV type I strain PT810, used as an example in this application. The present invention also contemplates BVDV virus-like particles comprising BVDV structural proteins which are not identical to naturally occurring proteins but contain amino acid substitutions, deletions and/or insertions provided that the mutant structural proteins retain the capability of being assembled into virus-like particles.

The BVDV structural proteins are derived from a polyprotein which is processed after translation. To prepare the BVDV virus-like particles according to the present invention it is favourable to use host cells which contain the information for synthesizing the structural proteins from a DNA template. A cDNA prepared from naturally occurring RNA which codes for the polyprotein of BVDV structural proteins would not work because mRNA transcribed from such a DNA template contains too many splice sites that would be recognised by spliceosomes in the cell nucleus. RNA transcribed from such cDNA would be destroyed before it can be translated.

The present invention, therefore, provides polycistronic RNA molecules comprising a ribonucleotide sequence which codes for the BVDV structural proteins N, E^{ms}, E1 and E2, which RNA is scarcely or not at all being spliced in the cell nucleus within its polyprotein encoding part.

A preferred embodiment of the RNA molecules according to the present invention is represented by RNA comprising a ribonucleotide sequence which codes for a poly-protein having the amino acid sequence according to SEQ ID NO: 2 and which does not contain strong potential splice sites within its polyprotein encoding part, i.e. no potential splice acceptor sites with a score above −22 and no potential splice donor sites with a score above −13.1.

A most preferred embodiment of the RNA molecules according to the present invention is represented by a RNA molecule which comprises a ribonucleotide sequence corresponding to the polynucleotide sequence from Nucleotide No. 17 to Nucleotide No. 2710 according to SEQ ID NO: 1.

The inventive RNA molecules can be obtained from corresponding DNA fragments which are also provided by the present invention.

The DNA fragments according to the present invention code for the BVDV structural proteins N, E^{ms}, E1 and E2, but do not contain strong potential splice sites. Splice sites are recognition sequences in eukaryotic mRNAs that are either exon-intron junctions (splice donor sequences) or intron-exon junctions (splice acceptor sequences) and are used by spliceosomes in the nucleus to remove introns from pre-mRNAs to fuse coding regions that are located on exons together to form a complete open reading frame. BVDV cDNA contains ‘accidental’ splicing signals that are never used because BVDV RNA normally stays in the cytoplasm. By expressing BVDV cDNA via viral vectors such as BHV1, the RNA will be made in the nucleus and be processed by the spliceosome [Shiu et al. (1997), The presence of RNA splicing signals in the cDNA construct of the E2 gene of classical swine fever virus affected its expression, J. Virol. Methods 69, 223-230].

To remove most of the (potential) splicing signals from the BVDV cDNA encoding N, E^{ms}, E1 and E2, the nucleic acid and protein sequence analysis software system of PC/Gene version 2.32 January 1989 can be used. In this software program the option “splice junctions” which is based on the method of Staden [(1984), Computer methods to locate signals in nucleic acid sequences, Nucl. Acids. Res. 12, 505-519] is appropriate to be used in the present invention.

A preferred embodiment of the DNA fragments according to the present invention is represented by a DNA comprising the polynucleotide sequence from Nucleotide No. 17 to Nucleotide No. 2710 according to SEQ ID NO: 1.

The present invention further provides DNA constructs suitable to produce BVDV virus-like particles. In order to allow the BVDV structural proteins to be expressed in host cells, the DNA is to be operably linked to cis-regulatory sequences. Such regulatory sequences are capable of binding RNA polymerases in a cell and of initiating transcription of a downstream (3′ direction) coding sequence. For purposes of defining the present invention, the cis-regulatory sequences are followed at the 3, terminus by a Kozak consensus sequence and the translation start codon (ATG) of a coding sequence and extend upstream (5′ direction) to include the minimum number of bases or elements necessary to initiate transcription at levels detectable above background. Within the cis-regulatory sequences a transcription initiation site as well as protein binding motifs responsible for the binding of RNA polymerase II complexes will be found. The choice of such regulatory sequences is obvious to those skilled in the art. Suitable cis-regulatory sequences can be derived from eukaryotic genes, preferably from mammals, or from genes from viruses that infect mammals. Examples are the simian virus 40 (SV40) promoter and the cis-regulatory sequences located on the long terminal repeats of retroviruses such as the murine leukemia virus (MuLV) long terminal repeat (MLV-LTR).

In a preferred embodiment of the DNA constructs according to the present invention the DNA encoding the polyprotein of BVDV structural proteins is operably linked to the human cytomegalovirus immediate early 1 promoter.

The DNA constructs according to the present invention preferably further comprise terminator sequences at the 3, terminus of the coding region. mRNA synthesis by RNA polymerase II in eukaryotic cells has a clear starting point, generally at a short distance downstream of the ‘TATA-box’, but no clear stop signal. The poly(A) signal found upstream of the 3′ end of the transcribed region plays an essential role in the cleavage and polyadenylation of the 3′ end of mRNAs [Keller (1995), No end yet to messenger RNA 3′ processing, Cell 81, 829-832] and is also implicated in termination of transcription, but sequences downstream of this poly(A) signal are thought to play a role in the termination of the mRNA transcription as well. These sequences are implicated in retarding or pausing the transcription complex [Vandenbergh (1991), An apparent pause site in the transcription unit of the rabbit alpha-globin gene, J. Mol. Biol. 220, 255-270]. As efficient termination and polyadenylation confers stability to mRNAs, termination signals—like the one found downstream of the bovine growth hormone gene—are often included in eukaryotic expression vectors.

In a preferred embodiment of the DNA constructs according to the present invention the DNA encoding the polyprotein of BVDV structural proteins is followed by the bovine growth hormone terminator sequence.

From a practical standpoint the use of viral vectors is favourable for the following reasons:

Viral vectors can express the proteins encoded on inserted gene(s) at high levels;

the recombinant viruses can be produced to high titres in suitable host cells at lower cost than subunit vaccines;

upon administration to the host the recombinant viruses express the proteins encoded on inserted gene(s), producing high amounts of these proteins in the host;

the proteins encoded by the inserted gene(s) are expressed intracellularly and can, therefore, be efficiently presented by MHC class I molecules to stimulate the cytotoxic T cells of the host;

all structural proteins of the BVDV genome can be expressed and form virus-like particles, comparable to modified live virus vaccine, but without the risks normally encountered with such vaccines, like e.g. reversion to wild-type;

live vaccines are generally more immunogenic than killed vaccines.

The present invention provides a viral vector encoding factors and BVDV structural proteins necessary for the assembly of BVDV virus-like particles.

In a preferred embodiment the viral vector is represented by bovine herpesvirus I (BHV-1).

In a further preferred embodiment a BHV-1 vector which contains a deletion within the sequence coding for glycoprotein B (gE) or which completely lacks the genome region coding for gE is used for preparing BVDV virus-like particles. An example for such a 13HV-1 vector is Difivac-1 deposited under Accession No. I-1213 with the Institut Pasteur, France. The use of such vector allows the production of a BHV-1/BVDV vaccine with double marker properties. Hosts, in particular cattle, immunised with the BHV-1/BVDV vaccine described herein form antibodies against BHV-1 and BVDV, but not against BHV-1 gE or the BHV-1gI/gE complex and not against the BVDV non structural proteins, in particular NS3 or p80. This allows discrimination of hosts, in particular cattle, infected with wild-type BHV-1 and/or wild-type BVDV from hosts only vaccinated with this BVH-1/BVDV vaccine, by measuring the presence of anti-BHV-1gE or anti-BHV-1/gE antibodies and/or anti-BVDV antibodies in their body fluids, in particular nasal fluid samples and serum samples.

A most preferred embodiment of the viral vector according to the present invention is represented by A9-SV-1F9 (synth. BVDV capsid; E^{ms}, E1 and E2 in gE locus of Difivac-1) deposited under CNCM accession No. 1-2488 with the Collection Nation-ale de Cultures de Microorganismes, Institut Pasteur (25, Rue du Docteur Roux, F-75724 Paris, France) on Jun. 8, 2000.

The present invention further provides host cells containing the above-mentioned viral vectors. The viral vectors can be introduced into the host cells by infection or transfection. Examples of suitable host cells are embryonic bovine trachea (EBTr) cells or Madin-Darby bovine kidney (MDBK) cells.

The present invention also provides a vaccine for immunising a host against BVDV induced diseases which comprises BVDV virus-like particles and a pharmaceutically acceptable carrier or diluent. Examples of pharmaceutically acceptable carriers or diluents useful in the present invention include stabilizers such as carbohydrates (e.g. sorbitol, mannitol, starch, sucrose, glucose, dextran), proteins such as albumin or casein, protein containing agents such as bovine serum or skimmed milk and buffers (e.g. phosphate buffer). Optionally, one or more compounds having adjuvant activity may be added to the vaccine. Suitable adjuvants are, for example, aluminium hydroxide, phosphate or oxide and oil emulsions such as saponins.

The useful effective amount to be administered will vary depending on age, weight, mode of administration and type of pathogen against which vaccination is sought. A suitable dosage can be, for example, about 10³-10⁷ pfu/animal.

The vaccine according to the present invention can be given inter alia intranasally, intradermally, subcutaneously or intramuscularly.

A preferred vaccine according to the present invention comprises a BHV-1/BVDV recombinant viral vector which will lead to the intracellular production of BVDV virus-like particles in the vaccinated host and will induce beneficial immune responses to protect against BVDV induced disease. Moreover, such a vaccine will also protect against BHV-1 induced disease and, if BHV-1 lacking gE is used as a vector, hosts vaccinated with such a vaccine can be discriminated from hosts infected either with BHV-1 or with BVDV.

The present invention further provides a diagnostic kit for detecting the presence and/or absence of anti-BVDV antibodies and, in particular, for detecting the presence and/or absence of anti-BVDV and anti-BHV-1 antibodies in a biological sample such as a biological fluid sample, in particular a nasal fluid sample or a serum sample. These diagnostic tests are used to discriminate between hosts infected with wild type virus and hosts only vaccinated with the BHV-1/BVDV vaccine described herein.

The anti-BHV-1 and/or anti-BVDV antibodies detected in these diagnostic tests are reacting with viral antigens that are not present in the vaccine, in particular BHV-1gE and the BHV-1 gI/gE complex and the BVDV non structural proteins, in particular NS3 orp80.

The present invention further provides a method for preparing BVDV virus-like particles comprising

(a) inserting a DNA construct as set forth above into a viral vector encoding factors for the assembly of BVDV virus-like particles,

(b) infecting suitable host cells capable of expressing the polyprotein encoded by the DNA, and

(c) culturing the host cells under appropriate conditions.

The present invention also provides a method for preparing BVDV virus-like particles comprising

(a) infecting suitable host cells with a viral vector as set forth above, and

(b) culturing the host cells under appropriate conditions.

The present invention further provides a method for preparing BHV1/BVDV recombinant viruses that encode all structural proteins for the formation of BVDV virus-like particles comprising

(a) inserting a DNA construct as set forth above into the genome of BHV-1,

(b) infecting suitable host cells capable of expressing the polyprotein encoded by the vector, and

(c) culturing the host cells under appropriate conditions.

Suitable host cells can be infected with the recombinant viruses by adding the supernatant of an infectious culture to a monolayer of these cells, e.g. EBTr cells or MDBK cells. For example, on a monolayer in a 150 cm² tissue culture flask, after removal of the medium, 1 to 3 ml of virus containing (titres ranging from 10³ to 10⁷ pfu/ml) culture medium can be added. After two hours incubation fresh tissue culture medium can be added up to 30 ml. Two to three days after infection full cytopathogenic effect (cpe) will be visible and up to 10⁸ pfu/ml can be found in the medium.

### EXAMPLES

Construction of the Coding Region for BVDV Structural Proteins

To determine the amino acid sequences of the structural proteins of a recent BVDV isolate, the 5′ half of the nucleotide sequence of BVDV type I strain PT810, which has recently been isolated in Europe, was established using standard methods.

The coding regions for the nucleocapsid protein N and the glycoproteins E^{ms}, E1 and E2 were identified by comparing the deduced amino acid sequences with published BVDV sequences. These coding regions contained sequences (splicing signals) which, when expressed by BHV-1 in the nucleus, could be recognized and processed by spliceosomes. Therefore, the sequence was adapted in such a way that most of the (potential) splicing signals were removed while the coding potential remained unaffected. The adapted sequence has been made synthetically and codes for the same amino acids as the original BVDV sequence with the exception of the first amino acid of the N protein.

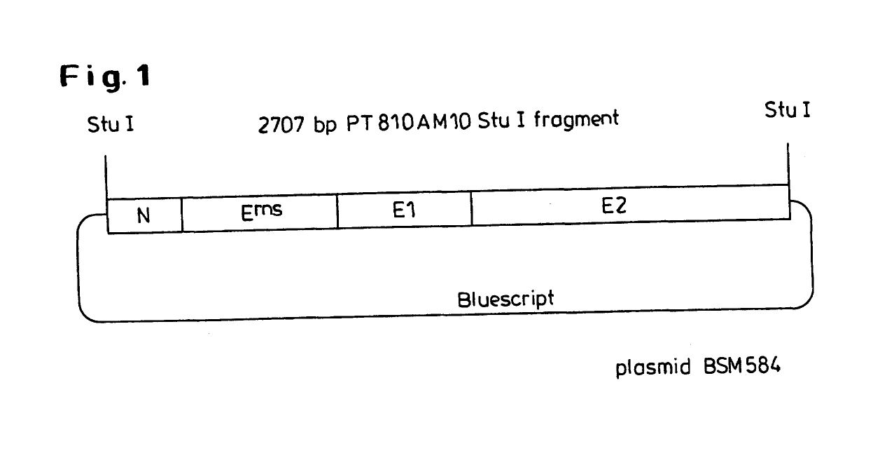

In BVDV strain PT810 the first amino acid of the N protein is a serine but in order to allow efficient translation of the synthetic coding region, this amino acid was replaced by a methionine and preceded by a Kozak consensus sequence. To end the protein encoded region, a stop codon was inserted behind the putative carboxy-terminal end of the E2 protein, right after its transmembrane region. To allow easy cloning, the sequence recognised by the Stu I restriction enzyme was added to both sides of the synthetic sequence bringing the total length to 2715 nucleotides. See SEQ ID NO: 1. A double stranded DNA fragment has been synthesised and cloned into prokaryotic plasmid Bluescript and propagated in Escherichia coli bacteria. See FIG. 1.

Construction of the BHV-1/BVDV Recombination/Expression Cassette for the gE Locus of Difivac-1

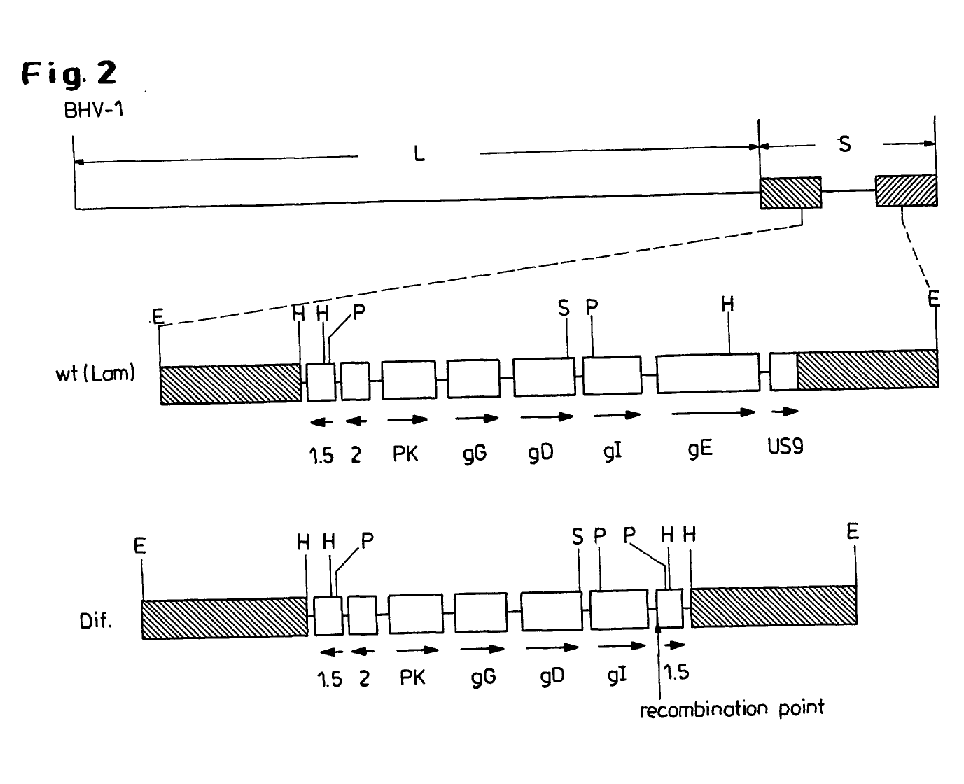

Analysis of the recombination found in the Uₛ region of the gE deleted Za strain (which has been renamed Difivac-1 in WO 92/21751) showed the deletion of the glycoprotein E gene and the neighbouring US9 gene and a concomitant duplication inversion of part of the US1.5 gene. This analysis also showed the position of the recombination point [Rijsewijk et al. (1999), Spontaneous BHV-1 recombinants in which the gI/gE/US9 region is replaced by a duplication/inversion of the US1.5/US2 region, Arch. Virol. 144, 1527-1537]. See FIG. 2.

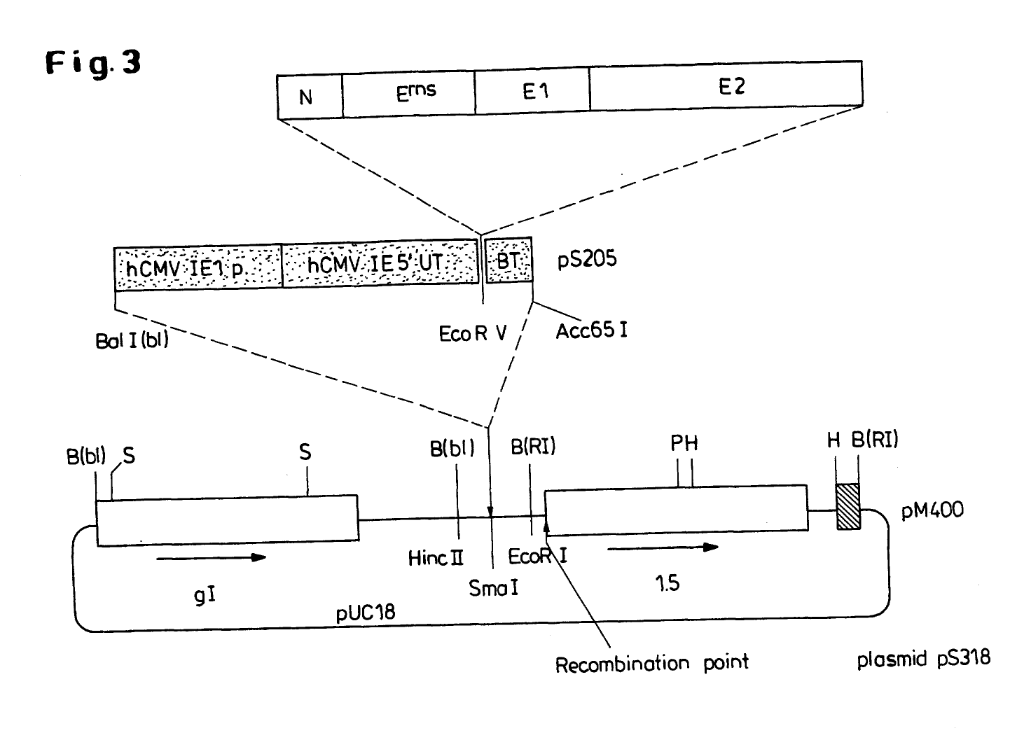

To insert heterologous genes in the genome of the Za strain at or close to the position of the gE deletion, a recombination cassette has been constructed. To this end, a 0.8 kilo base pair Bsa I fragment encoding part of the gI gene and ending 40 bp upstream of the Za recombination point and a 0.7 kilo base pair Bsa I fragment starting 40 bp downstream of the Za recombination point and encoding part of the US1.5 gene, have been cloned in pUC18 in their original orientation. This construct has been named pM400 and has between the recombination fragments a unique Sma I site for the insertion of the expression cassette. See FIG. 3.

In the Sma I site of pM400 a 1.9 kilo base pair Bal I-Acc65 I fragment from plasmid pVR1012 has been cloned with its cis-regulatory sequences in the same orientation as the flanking recombination fragments. These cis-regulatory sequences are the human cytomegalovirus immediate early 1 promoter (hCMVIEp.) and its 5′ untranslated leader (5′UT), and the bovine growth hormone terminator sequence (BT). The resulting construct has been named pS205 and has a unique EcoR V site between the promoter region and the terminator region.

In the EcoR V site of pS205 the 2707 base pair Stu I fragment, encoding the BVDV nucleocapsid protein N and the BVDV glycoproteins E^{ms}, E1 and E2, has been cloned in the same orientation as the promoter/terminator and the flanking recombination sequences. This BHV-1/BVDV recombination/expression cassette has been named pS318. See FIG. 3.

Construction and Isolation of BHV-1 Recombinant Virus Expression the Coding Region for BVDV Structural Proteins

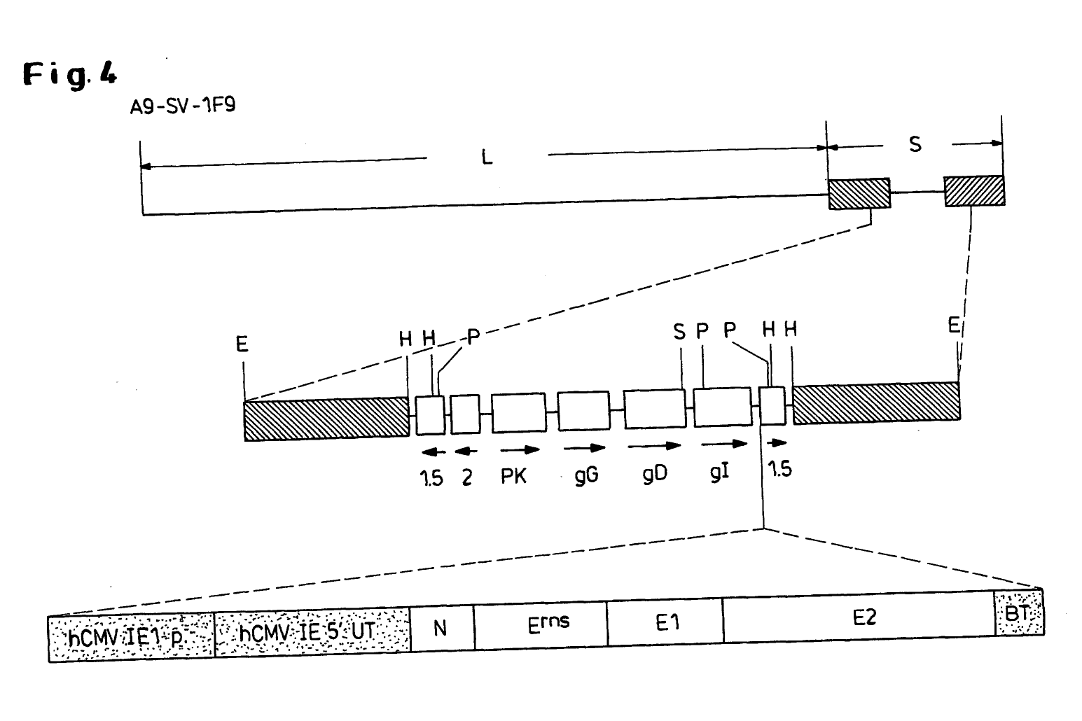

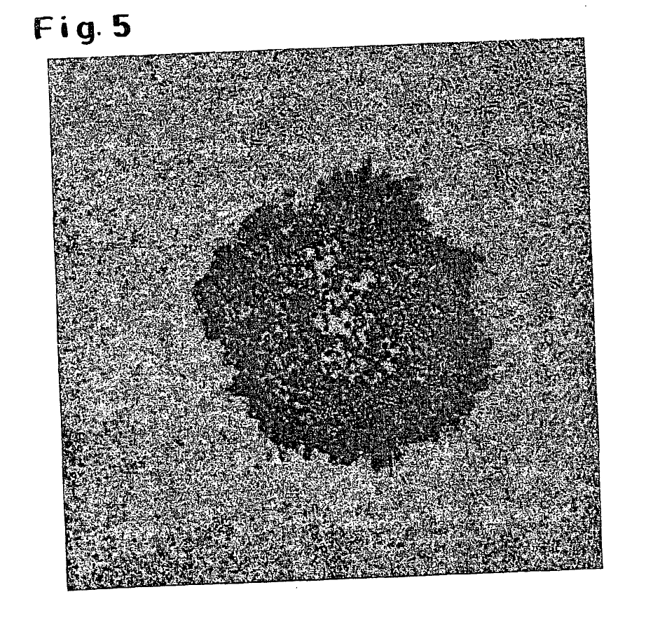

To construct the 3HV-1/BVDV recombinant (as described in FIG. 4) that expresses the BVDV nucleocapsid protein N and the BVDV glycoproteins E^{ms}, E1 and E2, linearized pS318 plasmid and genomic DNA of the Za strain were cotransfected into bovine cells according to standard methods and 48 hours after cotransfection cells were freeze/thawed to liberate putative recombinant viruses. To isolate these recombinant viruses, the cell debris was pelleted and the supernatant was used to infect bovine cells on a 96 wells plate and to identify BVDV positive wells using an immunostaining procedure with anti-BVDV E2 MAb 166. The virus in the medium of BVDV E2 positive wells was diluted to infect bovine cells on another 96 wells microplate and virus from BVDV E2 positive single plaques was three times plaque purified to obtain a purified recombinant. One of the recombinants obtained by this way was named A9-SV-1F9 and passaged 8 times on MDBK cells to test the stability of the expression on a panel of six different bovine cell lines. See FIG. 5. In all bovine cell types tested, even after 8 passages, virtually all plaques were found BVDV E2 positive. In MDBK cells the percentage of BVDV E2 positive plaques was determined and found to be still more than 90% after 8 passages.

Legends to Sequence Listing and Figures

SEQ ID NOs: 1 and 2

The 2715 nucleotides long sequence of the synthetic DNA fragment PT810AM10 which codes for proteins N, E^{ms}, E1 and E2 of BVDV strain PT810 is shown. To both sides of the DNA fragment the recognition sequence of restriction enzyme Stu I is added and upstream of the start codon (ATG) a Kozak consensus sequences is inserted. The PT810AM10 sequence has been translated using the universal genetic code and the encoded amino acid sequence has been indicated in the three letter code below the nucleotide sequence. All amino acids encoded by the open reading frame are identical to the ones found in BVDV strain PT810, with exception of the first amino acid of the open reading frame. This first amino acid is a serine residue in PT810 and was changed into a methionine residue in PT810AM10. The stop codon at the end of the protein coding region overlaps with the Stu I site at the end of the DNA fragment.

SEQ ID NOs: 3 and 4

Segment of SEQ ID NO: 1 coding for protein N.

SEQ ID NOs: 5 and 6

Segment of SEQ ID NO: 1 coding for protein En.

SEQ ID NOS: 7 and 8

Segment of SEQ ID NO: 1 coding for protein E1.

SEQ ID NOs: 9 and 10

Segment of SEQ ID NO: 1 coding for protein E2.

FIG. 1

Structure of plasmid BSM584. The 2715 nucleotides long DNA fragment PT810AM10 has been cloned in prokaryotic plasmid Bluescript. By digesting plasmid BSM584 with restriction enzyme Stu I a 2707 nucleotides long fragment was liberated that was inserted into the BHV-1 recombination/expression cassette pS205. See FIG. 3.

FIG. 2

Top: Diagram of the structure of the BHV-1 genome and the recombination found in the Za or Difivac-1 strain that has been used as a vector. Wild type BHV-1 is approximately 136 kilo base pares long and has a Long (L) and a short (S) segment. The short fragment has a unique domain bordered by an inverted repeat indicated by hatched boxes.

Middle: In unique short (U3s) domain 8 open reading frames have been recognised: US1.5, US2, PK, gG, gD, gL gE and US9.

Bottom: In the natural gE deletion mutant Za or Difivac-1 (Dif.) the gE and US9 genes have been deleted and part of the US 1.5 gene has been duplicated instead. The recombination point has been indicated by an arrow. This Difivac-1 mutant has been described in WO 92/21751 and in Arch. Virol. (1999) 144, 1527-1537 by Rijsewijk et al.

FIG. 3

Diagram of the recombination/expression cassette plasmid pS318. The 0.8 kilo base pairs Bsa I (B) fragment from the upstream side of the gE locus and the 0.7 kilo base pairs Bsa I fragment from the downstream side of the gE locus, both isolated from the Difivac-1 strain, have been cloned in respectively the Hinc II site and the EcoR I site of prokaryotic plasmid pUC18. To insert the 0.8 kbp Bsa I fragment into the Hinc II site the Bsa I fragment has been made blunt ended and to insert the 0.7 kbp Bsa I fragment into the EcoR I site EcoR I linkers have been added to this fragment using standard methods. The resulting plasmid has been named pM400. In the Sma I site of pM400 a fragment taken from plasmid pVR1012 containing the IE1 promoter/enhancer of the human cytomegalovirus (hCMVIEp.) and the 5′ untranslated region of the same promoter (5′UT) and the bovine growth hormone terminator (BT) sequence, has been inserted. The resulting plasmid has been named pS205. Into the unique EcoR V site of pS205 the 2707 bp Sta I fragment from plasmid BSM584 has been inserted. The resulting clone has been named plasmid pS318 and this plasmid has been used in cotransfection experiments to recombine the BVDV genes into the gE locus of Za (Difivac-1). See FIG. 4.

FIG. 4

Diagram of the BHV-1/BVDV recombinant A9-SV-1F9 that encodes all structural proteins of BVDV strain PT810. On the top the structure of the BHV-1 genome has been indicated. In the middle the EcoR I fragment that includes the unique short region with the position of all genes and on the bottom the BVDV expression cassette with the 2707 bp long fragment encoding the structural proteins of BVDV stain PT810 have been indicated. The BVDV open reading frame has been preceded by the human cytomegalovirus IE1 promoter/enhancer region (hCMVIE1p.) and the 5′ untranslated (5′UT) leader of this promoter and the BVDV open reading frame has been followed by the bovine growth hormone terminator sequence (BT). The cassette has been inserted into the Difivac-1 genome 40 bp upstream of the recombination site found in this genome at the gE locus in the same orientation as the surrounding genes.

FIG. 5

IPMA of a monolayer of MDBK cells infected with BHV-1/BVDV recombinant A9-SV-1F9 and stained with anti- BVDV E2 MAb 166 four days after infection. The infected cells form a round plaque that is stained by the antibody while the surrounding cells are not stained.

1. BVDV virus-like particles.

2. BVDV virus-like particles according to claim 1 comprising the BVDV structural proteins N, E^{ms}, E1 and E2.

3. Polycistronic RNA molecule comprising a ribonucleotide sequence encoding a polyprotein consisting of the BVDV structural proteins N, E^{ms}, E1 and E2, said RNA molecule being not spliced in the cell nucleus within its poly-protein encoding part.

4. RNA molecule according to claim 3 encoding a polyprotein having the amino acid sequence according to SEQ ID NO: 2 provided that said RNA molecule does not contain strong potential splice sites.

5. RNA molecule according to claim 4 comprising a ribonucleotide sequence corresponding to the polynucleotide sequence from Nucleotide No. 17 to Nucleotide No. 2710 according to SEQ ID NO: 1

6. DNA fragment corresponding to the RNA molecule according to any of claims 3 to 5.

7. DNA fragment according to claim 6 comprising the polynucleotide sequence from Nucleotide No. 17 to Nucleotide No. 2710 according to SEQ ID NO: 1.

8. DNA construct comprising the DNA according to claim 6 or 7 operably linked to cis-regulatory sequences capable of controlling the expression of the polyprotein encoded by said DNA.

9. DNA construct according to claim 8 further comprising a terminator sequence.

10. DNA construct according to claim 8 or 9 wherein the cis-regulatory sequences are derived from the human cytomegalovirus immediate early 1 promoter and 5′ untranslated leader.

11. DNA construct according to claim 9 wherein the terminator sequence is derived from the bovine growth hormone terminator sequence.

12. Viral vector encoding factors for the assembly of BVDV virus-like particles, said viral vector comprising the DNA construct according to any of claims 8 to 11.

13. Viral vector according to claim 12 which is BHV-1 or a BHV-1 deletion mutant.

14. Viral vector according to claim 13 which is Difivac-1 deposited under Accession No. I-1213.

15. Viral vector according to any of claims 12 to 14 wherein said BHV-1 vector carries said DNA construct within the sequence coding for glycoprotein gE or at the position within a mutant BHV-1 vector where the sequence coding for glycoprotein gE is deleted.

16. Viral vector according to claim 15 which is A9-SV-1F9 deposited under the CNCM accession No. I-2488.

17. Host cell containing the vector according to any of claims 12 to 16.

18. Vaccine comprising BVDV virus-like particles according to claim 1 or 2 and a pharmaceutically acceptable carrier or diluent.

19. Vaccine according to claim 18 father comprising BHV-1.

20. Vaccine according to claim 19 wherein said BHV-1 lacks glycoprotein gE.

21. Vaccine according to claim 20 wherein said BHV-1 is Difivac-1 deposited under CNCM accession No. I-1213.

22. Vaccine comprising recombinant viruses encoded by and including the vector according to any of claims 12 to 16 and a pharmaceutically acceptable carrier or diluent.

23. Diagnostic kit containing BVDV virus-like particles according to claim 1 or 2.

24. Diagnostic kit according to claim 23 further containing BVDV NS3 and/or BVDV p80 protein or immunogenic fragments thereof.

25. Diagnostic kit according to claim 23 or 24 further containing BHV-1 gE protein and/or BHV-1 gI/gE protein complex or immunogenic fragments thereof.

26. Method for preparing BVDV virus-like particles comprising
(a) inserting the DNA construct according to any of claims 8 to 11 into a viral vector encoding factors for the assembly of BVDV virus-like particles,
(b) infecting suitable host cells capable of expressing the polyprotein en- coded by said DNA, and
(c) culturing said host cells under appropriate conditions.

27. Method for preparing recombinant viruses encoded by and including the vector according to any of claims 12 to 16 comprising
(a) infecting suitable host cells with a viral vector according to 12 to 16,
(b) culturing said host cells under appropriate conditions, and optionally
(c) isolating the recombinant viruses.

28. Method for preparing a vaccine according to claim 22 comprising admixing the recombinant viruses with a pharmaceutically acceptable carrier.


SEQUENCE LISTING (10 sequences)

SEQ ID NO: 1 - DNA, 2715 bp, Bovine Viral Diarrhea Virus
  3′UTR (8)..(16)
  3′UTR (8)..(16): Kozak Consensus Sequence
aaggcctgccgccaccatggacacgaaagaagaaggggcaacaaaaaaacaacagaaaccggaccgcgttgaaaaagggcgcatgaaaataacgcctaaagaaactgaaaaagattcccggaccaaaccacctgatgctacgatcgtcgtcgacggcgtcaaataccaagtcaaaaaaaaaggcaaagtcaaatccaaaaacacccaagatgggctctaccacaataaaaataaaccacaagaatcacgcaaaaaactggaaaaagctctattggcttgggcaatattggctgttgtattatttcaagtcacaatgggggaaaacataacacaatggaacttgcaagacaatggaaccgaaggcgtccaacgggctatgtttgaacgcggcgtcaatcggagcttacatggaatctggcccgaaaaaatctgcaccggcgtcccatctcatttggccaccgatatggaattgaaacgaattcatggaatgatggacgcatctgaaaaaaccaactatacatgctgccggcttcaacgacatgaatggaataaacatggctggtgcaattggtacaatatcgaaccttggattctgcttatgaatcggacccaagctaacctcactgaaggccaaccacaacgcgaatgcgccgtcacctgccgctatgaccggaattccgacttgaatgtcgtgacacaagcccgggactctccgacaccacttacgggatgcaaaaaagggaaaaacttctctttttcgggcatcgtcatccaaggcccttgcaattttgaaattgctgcatctgacgtcctcttcaaagaacatgactgcacatccatatttcaagatactgctcattacctcgttgatgggatgactaactctttggagtctgctcgacaaggaactgcaaaactaacaacttggctggggcgacaacttgggatattggggaaaaaactggaaaacaaatccaaaacatggttcggggcttatgcatcttccccttactgcgatgttgaacgaaaacttggctacatctggtttacaaaaaattgcacccctgcctgcctccccaaaaacacaaaaattgttggacctgggaaatttgacaccaatgccgaagatggaaaaatattacatgaaatggggggccacctttcggaagttctattactttcacttgttgttctatccgatttcgcacccgaaactgcctctgcgatgtatcttgtcctacatttttccatcccacaacgacacaccgatgttctggactgcgataaatctcaactaaatctaaccatgggcgtcacaaccgccgatgttatacccggatccgtctggaatatgggcaaatatgtttgcatacgacccgactggtggccttatgaaacggctgctgttctggctttggaagaagttgggcaagttacacggatcgtcttgcgggcactccgcgacttgacacgcatctggaacgctgccacaaccactgcatttcttgtctgccttgttaaagttgtccgcggacaagtcttacaaggcgtcatatggttactgctaataacgggcgtccaaggacgcctcgattgcaaacctgacttctcatatgccattgccaaaaatgaaaaaattggaccactgggggctgaaggacttactaccacttggtatgaatactctgatgggatgcaactttccgacactatggttgaagctcgatgcaaagatggggaatttacattcatccaaaaatgcaaaacggaaacccgatatctggccaccttgcacacacgggccttaccgacatctgtcgtttttgaaaaactttttgatggaaataaattggcggacatcgttgaaatggatgacaacttcgaatttgcgatctgcccctgcgatgcaaaacccgtcgtccgcgggaaatttaacacaacactactaaatgggcccgccttccaaatggtctgccccattggatggactggatctgtctcctgcaccctagccaataaagacaccctcgatacggccgtcgtccggacatataaacgcgtttccccattccctaatcggcaaggatgcgttactcaaaaacttctcggggaagatctttatgattgcatcttgggcggaaactggacttgcatcgaaggggaacaactacgatacactgggggcaccattgaatcctgcaagtggtgcggctacaaattcttgaaatcggaagggctaccacactatccaattggcaaatgccgcttacaaaatgaaactggctaccggcttgtcgacgacacctcttgcaatgtcggcggcgtcgcaattgtcccacatggacttgtcaaatgcaaaattggggataccgtcgtccaagtcgtcgcaatggacacgaaacttggacctatgccttgcaaaccacatgaaataatatcatcggaaggacccgttgaaaaaacggcatgcacattcaactatacacggaccttaacgaacaaatattttgaaccccgggacaattacttccaacaatacatgctaaaaggggactaccaatattggtttgatctggaagtctctgaccaccatcgggattactttacggaattcctacttgtcattgttgtcgccctcttgggcggacgctatgtcctttggctacttgtcacatacatggtcctctccgaacaaaatgcctcggcttaggcctt

SEQ ID NO: 2 - protein, 897 aa, Bovine Viral Diarrhea Virus
MDTKEEGATKKQQKPDRVEKGRMKITPKETEKDSRTKPPDATIVVDGVKYQVKKKGKVKSKNTQDGLYHNKNKPQESRKKLEKALLAWAILAVVLFQVTMGENITQWNLQDNGTEGVQRAMFERGVNRSLHGIWPEKICTGVPSHLATDMELKRIHGMMDASEKTNYTCCRLQRHEWNKHGWCNWYNIEPWILLMNRTQANLTEGQPQRECAVTCRYDRNSDLNVVTQARDSPTPLTGCKKGKNFSFSGIVIQGPCNFEIAASDVLFKEHDCTSIFQDTAHYLVDGMTNSLESARQGTAKLTTWLGRQLGILGKKLENKSKTWFGAYASSPYCDVERKLGYIWFTKNCTPACLPKNTKIVGPGKFDTNAEDGKILHEMGGHLSEVLLLSLVVLSDFAPETASAMYLVLHFSIPQRHTDVLDCDKSQLNLTMGVTTADVIPGSVWNMGKYVCIRPDWWPYETAAVLALEEVGQVTRIVLRALRDLTRIWNAATTTAFLVCLVKVVRGQVLQGVIWLLLITGVQGRLDCKPDFSYAIAKNEKIGPLGAEGLTTTWYEYSDGMQLSDTMVEARCKDGEFTFIQKCKTETRYLATLHTRALPTSVVFEKLFDGNKLADIVEMDDNFEFAICPCDAKPVVRGKFNTTLLNGPAFQMVCPIGWTGSVSCTLANKDTLDTAVVRTYKRVSPFPNRQGCVTQKLLGEDLYDCILGGNWTCIEGEQLRYTGGTIESCKWCGYKFLKSEGLPHYPIGKCRLQNETGYRLVDDTSCNVGGVAIVPHGLVKCKIGDTVVQVVAMDTKLGPMPCKPHEIISSEGPVEKTACTFNYTRTLTNKYFEPRDNYFQQYMLKGDYQYWFDLEVSDHHRDYFTEFLLVIVVALLGGRYVLWLLVTYMVLSEQNASA

SEQ ID NO: 3 - DNA, 303 bp, Bovine Viral Diarrhea Virus
  CDS (1)..(303)
atggacacgaaagaagaaggggcaacaaaaaaacaacagaaaccggaccgcgttgaaaaagggcgcatgaaaataacgcctaaagaaactgaaaaagattcccggaccaaaccacctgatgctacgatcgtcgtcgacggcgtcaaataccaagtcaaaaaaaaaggcaaagtcaaatccaaaaacacccaagatgggctctaccacaataaaaataaaccacaagaatcacgcaaaaaactggaaaaagctctattggcttgggcaatattggctgttgtattatttcaagtcacaatgggg

SEQ ID NO: 4 - protein, 101 aa, Bovine Viral Diarrhea Virus
MDTKEEGATKKQQKPDRVEKGRMKITPKETEKDSRTKPPDATIVVDGVKYQVKKKGKVKSKNTQDGLYHNKNKPQESRKKLEKALLAWAILAVVLFQVTMG

SEQ ID NO: 5 - DNA, 681 bp, Bovine Viral Diarrhea Virus
  CDS (1)..(681)
gaaaacataacacaatggaacttgcaagacaatggaaccgaaggcgtccaacgggctatgtttgaacgcggcgtcaatcggagcttacatggaatctggcccgaaaaaatctgcaccggcgtcccatctcatttggccaccgatatggaattgaaacgaattcatggaatgatggacgcatctgaaaaaaccaactatacatgctgccggcttcaacgacatgaatggaataaacatggctggtgcaattggtacaatatcgaaccttggattctgcttatgaatcggacccaagctaacctcactgaaggccaaccacaacgcgaatgcgccgtcacctgccgctatgaccggaattccgacttgaatgtcgtgacacaagcccgggactctccgacaccacttacgggatgcaaaaaagggaaaaacttctctttttcgggcatcgtcatccaaggcccttgcaattttgaaattgctgcatctgacgtcctcttcaaagaacatgactgcacatccatatttcaagatactgctcattacctcgttgatgggatgactaactctttggagtctgctcgacaaggaactgcaaaactaacaacttggctggggcgacaacttgggatattggggaaaaaactggaaaacaaatccaaaacatggttcggggcttatgca

SEQ ID NO: 6 - protein, 227 aa, Bovine Viral Diarrhea Virus
ENITQWNLQDNGTEGVQRAMFERGVNRSLHGIWPEKICTGVPSHLATDMELKRIHGMMDASEKTNYTCCRLQRHEWNKHGWCNWYNIEPWILLMNRTQANLTEGQPQRECAVTCRYDRNSDLNVVTQARDSPTPLTGCKKGKNFSFSGIVIQGPCNFEIAASDVLFKEHDCTSIFQDTAHYLVDGMTNSLESARQGTAKLTTWLGRQLGILGKKLENKSKTWFGAYA

SEQ ID NO: 7 - DNA, 585 bp, Bovine Viral Diarrhea Virus
  CDS (1)..(585): Protein E1
tcttccccttactgcgatgttgaacgaaaacttggctacatctggtttacaaaaaattgcacccctgcctgcctccccaaaaacacaaaaattgttggacctgggaaatttgacaccaatgccgaagatggaaaaatattacatgaaatggggggccacctttcggaagttctattactttcacttgttgttctatccgatttcgcacccgaaactgcctctgcgatgtatcttgtcctacatttttccatcccacaacgacacaccgatgttctggactgcgataaatctcaactaaatctaaccatgggcgtcacaaccgccgatgttatacccggatccgtctggaatatgggcaaatatgtttgcatacgacccgactggtggccttatgaaacggctgctgttctggctttggaagaagttgggcaagttacacggatcgtcttgcgggcactccgcgacttgacacgcatctggaacgctgccacaaccactgcatttcttgtctgccttgttaaagttgtccgcggacaagtcttacaaggcgtcatatggttactgctaataacgggcgtccaagga

SEQ ID NO: 8 - protein, 195 aa, Bovine Viral Diarrhea Virus
SSPYCDVERKLGYIWFTKNCTPACLPKNTKIVGPGKFDTNAEDGKILHEMGGHLSEVLLLSLVVLSDFAPETASAMYLVLHFSIPQRHTDVLDCDKSQLNLTMGVTTADVIPGSVWNMGKYVCIRPDWWPYETAAVLALEEVGQVTRIVLRALRDLTRIWNAATTTAFLVCLVKVVRGQVLQGVIWLLLITGVQG

SEQ ID NO: 9 - DNA, 1122 bp, Bovine Viral Diarrhea Virus
  CDS (1)..(1122): Protein E2
cgcctcgattgcaaacctgacttctcatatgccattgccaaaaatgaaaaaattggaccactgggggctgaaggacttactaccacttggtatgaatactctgatgggatgcaactttccgacactatggttgaagctcgatgcaaagatggggaatttacattcatccaaaaatgcaaaacggaaacccgatatctggccaccttgcacacacgggccttaccgacatctgtcgtttttgaaaaactttttgatggaaataaattggcggacatcgttgaaatggatgacaacttcgaatttgcgatctgcccctgcgatgcaaaacccgtcgtccgcgggaaatttaacacaacactactaaatgggcccgccttccaaatggtctgccccattggatggactggatctgtctcctgcaccctagccaataaagacaccctcgatacggccgtcgtccggacatataaacgcgtttccccattccctaatcggcaaggatgcgttactcaaaaacttctcggggaagatctttatgattgcatcttgggcggaaactggacttgcatcgaaggggaacaactacgatacactgggggcaccattgaatcctgcaagtggtgcggctacaaattcttgaaatcggaagggctaccacactatccaattggcaaatgccgcttacaaaatgaaactggctaccggcttgtcgacgacacctcttgcaatgtcggcggcgtcgcaattgtcccacatggacttgtcaaatgcaaaattggggataccgtcgtccaagtcgtcgcaatggacacgaaacttggacctatgccttgcaaaccacatgaaataatatcatcggaaggacccgttgaaaaaacggcatgcacattcaactatacacggaccttaacgaacaaatattttgaaccccgggacaattacttccaacaatacatgctaaaaggggactaccaatattggtttgatctggaagtctctgaccaccatcgggattactttacggaattcctacttgtcattgttgtcgccctcttgggcggacgctatgtcctttggctacttgtcacatacatggtcctctccgaacaaaatgcctcggct

SEQ ID NO: 10 - protein, 374 aa, Bovine Viral Diarrhea Virus
RLDCKPDFSYAIAKNEKIGPLGAEGLTTTWYEYSDGMQLSDTMVEARCKDGEFTFIQKCKTETRYLATLHTRALPTSVVFEKLFDGNKLADIVEMDDNFEFAICPCDAKPVVRGKFNTTLLNGPAFQMVCPIGWTGSVSCTLANKDTLDTAVVRTYKRVSPFPNRQGCVTQKLLGEDLYDCILGGNWTCIEGEQLRYTGGTIESCKWCGYKFLKSEGLPHYPIGKCRLQNETGYRLVDDTSCNVGGVAIVPHGLVKCKIGDTVVQVVAMDTKLGPMPCKPHEIISSEGPVEKTACTFNYTRTLTNKYFEPRDNYFQQYMLKGDYQYWFDLEVSDHHRDYFTEFLLVIVVALLGGRYVLWLLVTYMVLSEQNASA